(19) United States
(12) Patent Application Publication
(10) Pub. No.: US 2005/0230408 A1
(43) Pub. Date: Oct. 20, 2005

(54) Single dispensing wetted tissue box

(76) Inventor: Richard H. Seager, Mystic, CT (US)

Correspondence Address: BACHMAN & LAPOINTE, P.C., 900 CHAPEL STREET, SUITE 1201, NEW HAVEN, CT, 06510, US

(21) Appl. No.: 11/056,586

(22) Filed: Feb. 11, 2005

Related U.S. Application Data
(60) Provisional application No. 60/544,297, filed on Feb. 11, 2004.

(51) Int. Cl. A47K 10/24; B65H 1/00; A61B 17/00

(52) U.S. Cl. 221/45

(57) ABSTRACT

A package for dispensing tissues including a body having a lower body portion and an upper body portion defining a tissue holding space, wherein the lower body portion is hingedly connected to the upper body portion along a first pivot axis, and wherein the upper body portion has a tissue opening for allowing tissues to be removed from the tissue holding space, and a tissue access hatch hingedly connected to the body along a second pivot axis which is spaced from and substantially parallel to the first pivot axis.

### CROSS-REFERENCE TO RELATED APPLICATION

This application claims the benefit of U.S. Provisional Application Ser. No. 60/544,297, filed Feb. 11, 2004.

### BACKGROUND OF THE INVENTION

The invention relates to a single dispensing wetted tissue box and, more particularly, to such a box designed for travel applications.

Various different types of tissue dispensing boxes are known in the art. These boxes are typically designed in an attempt to provide ease in removal of a single tissue at a time, ease in opening of the package, and efficient manufacture. Despite these important goals, the need remains for dispensing tissue boxes which present desirable characteristics in each of these areas.

It is therefore the primary object of the present invention to provide a single dispensing tissue box which accomplishes the aforesaid objectives.

Other objects and advantages of the present invention will appear hereinbelow.

### SUMMARY OF THE INVENTION

According to the invention, a single dispensing wetted tissue box has been provided which accomplishes the aforesaid objectives.

According to the invention, a tissue box is provided which has a double-hinged structure whereby the main body has a hinge and latch structure to allow for opening to load with a stack of tissues, while the upper portion of the main body has a hinge with a tissue access hatch which is used to access and remove tissues one at a time.

The invention can be formed in a single piece to enhance manufacture in this manner, or can be made in two pieces so as to reduce the footprint of the mold in the injection molding machine.

Still further, a package for dispensing tissues comprises a body having a lower body portion and an upper body portion defining a tissue holding space, wherein the lower body portion is hingedly connected to the upper body portion along a first pivot axis, and wherein the upper body portion has a tissue opening for allowing tissues to be removed from the tissue holding space, and a tissue access hatch hingedly connected to the body along a second pivot axis which is spaced from and substantially parallel to the first pivot axis.

### BRIEF DESCRIPTION OF THE DRAWINGS

A detailed description of preferred embodiments of the present invention follows, with reference to the attached drawings, wherein:

FIG. 1 illustrates a tissue box in accordance with the present invention;

FIG. 2 illustrates the tissue box of FIG. 1 with the tissue hatch open;

FIG. 3 illustrates the tissue box of FIG. 1 with both the main body and the tissue hatch open;

FIG. 4 illustrates loading of a tissue stack into a tissue box in accordance with the present invention;

FIG. 5 illustrates an as molded part in accordance with the embodiment of FIG. 1;

FIG. 6 illustrates an alternative embodiment in accordance with the present invention;

FIG. 7 further illustrates the embodiment of FIG. 6;

FIG. 8 further illustrates the embodiment of FIG. 6;

FIGS. 9 and 10 illustrate assembly of the tissue hatch to the main body in accordance with the embodiment of FIG. 6;

FIG. 11 illustrates the as molded parts for the embodiment of FIG. 6; and

FIG. 12 illustrates loading of the embodiment of FIG. 6.

### DETAILED DESCRIPTION

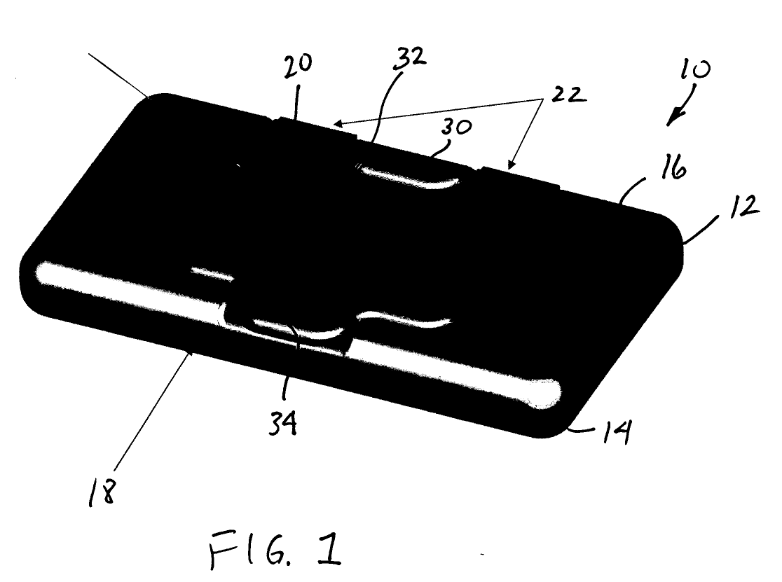

The invention relates to a single dispensing wetted tissue box. Referring to the drawings, FIG. 1 shows a box 10 in accordance with the present invention having a main body 12 defined by a lower body portion 14 and an upper body portion 16. Lower body portion 14 and upper body portion 16 are connected by a main body hinge 18 and a latch mechanism which is not shown in FIG. 1, to define a body for holding tissues such as wetted tissue wipes and the like.

A tissue access hatch 20 is also provided and is connected to upper body portion 16 by hatch hinges 22 to allow for pivot of tissue access hatch 20 relative to upper body portion 16 and subsequent access to tissues contained within the package.

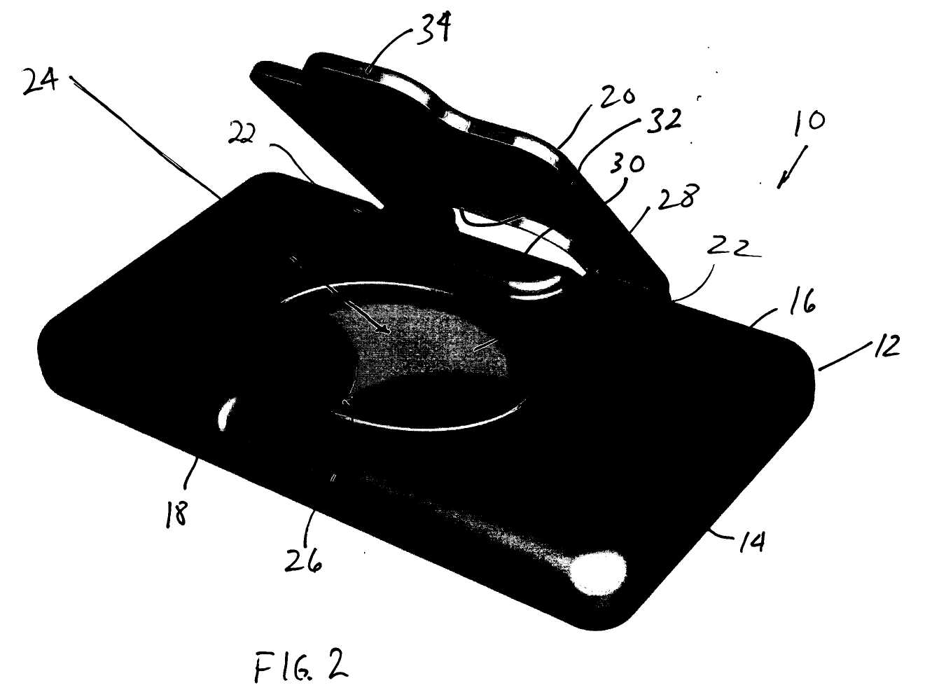

FIG. 2 shows package 10 with tissue access hatch 20 open. As shown, when tissue access hatch 20 is open, a tissue access area 24 is exposed which is adapted to allow for removal of tissues by a user, preferably one tissue at a time with ease of separation of same. In the embodiment shown in FIG. 2, this is accomplished by providing an opening having a specific contour including a restricted area or throat 26 which increases hold on a tissue so that tissues separate when one tissue and a subsequent tissue clinging thereto are pulled through throat 26. The larger open area 28 of tissue access area 24 advantageously allows for ease of grasping of an exposed edge of a tissue lying thereunder. This structure of a dispensing opening profile area is advantageously selected so as to provide ease in removal of a single tissue from within box 10. Of course, other shapes and styles of opening profiles could be provided and would be well within the scope of the present invention.

As shown, tissue hatch 20 is hinged to upper body portion 16 by two hinges 22. In this embodiment, these hinges are split and an upwardly projecting portion 30 is positioned to extend upwardly through a cutout 32 in tissue hatch 20.

It should also be noted that a detent latch is advantageously located under the finger lift 34 of tissue hatch 20 for ease in opening the package.

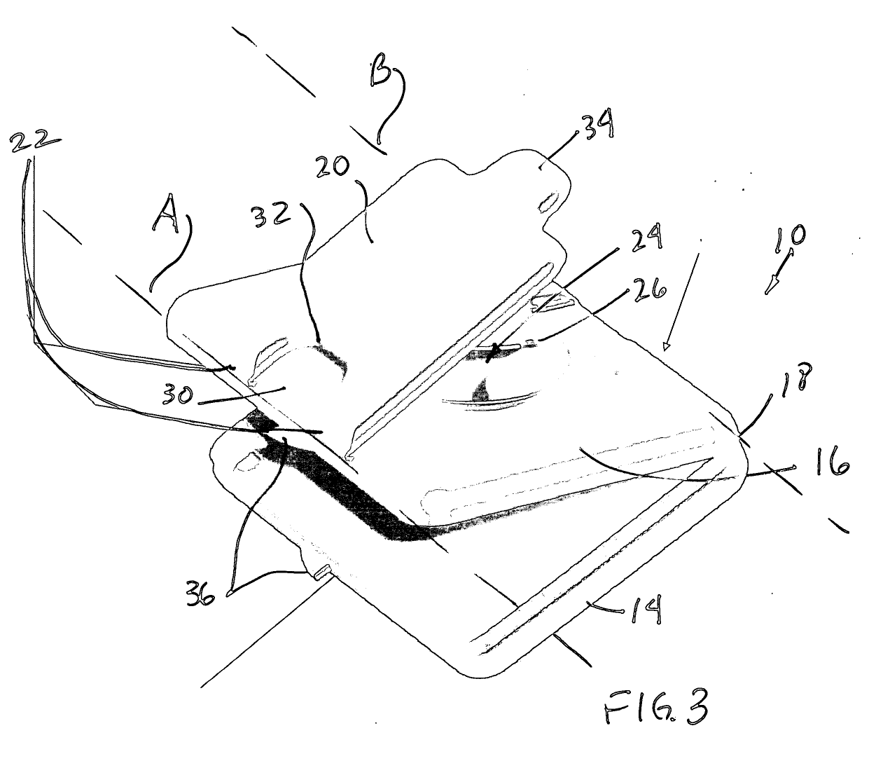

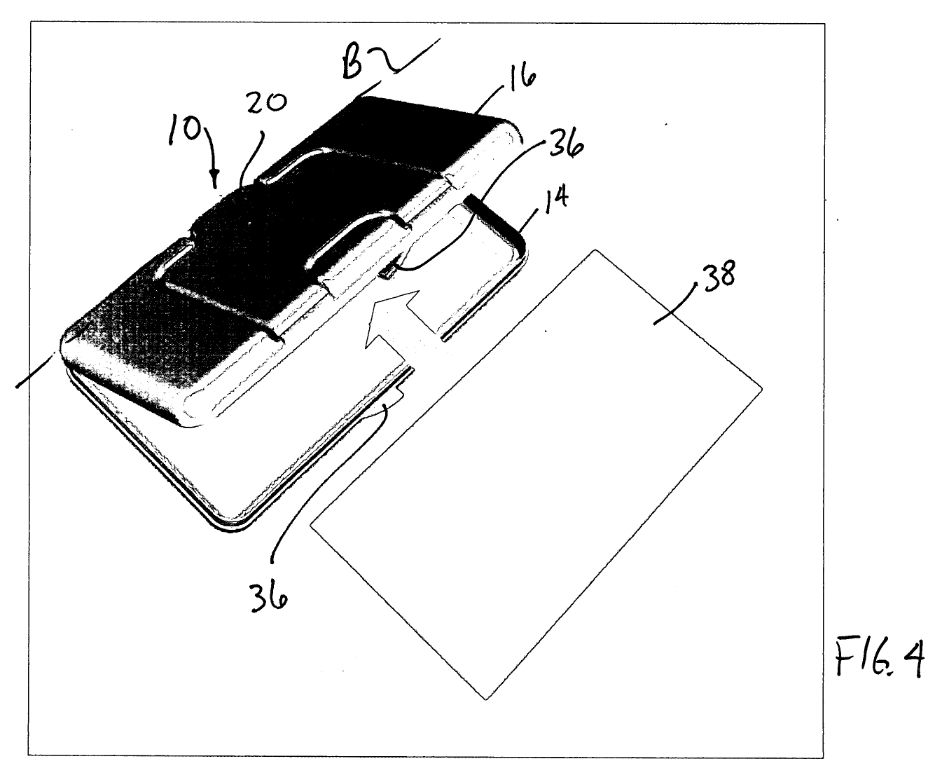

Turning to FIG. 3, box 10 is shown with main body portion 16 opened relative to lower body portion 14 and with tissue hatch 20 opened relative to upper body portion 16. This illustrates the opposite location of the two different hinge points in accordance with the present invention. That is, tissue hatch 20 is advantageously hinged to upper body portion 16 such that tissue hatch 20 can pivot relative to upper body portion 16 around an axis A which passes along hinges 22. Upper body portion 16 is advantageously hinged to lower body portion 14 such that upper body portion 16 can pivot relative to lower body portion 14 along an axis B (see also FIG. 4), and axes A and B are advantageously substantially parallel to and spaced from each other.

A latch 36 is shown on upper body portion 16 and lower body portion 14 for securing these portions in a closed position. This latch is shown as a pair of engaging structures one on each body portion, which engage in the closed position. This latch mechanism is advantageously constructed based upon the premise that the main body is only opened for loading of tissue stacks, and is not typically opened for single wipe dispensing.

FIG. 4 shows box 10 in accordance with the present invention with upper body portion 16 open relative to lower body portion 14 to allow for introduction of a new tissue stack 38 into box 10. In this manner, box 10 is reusable and refillable.

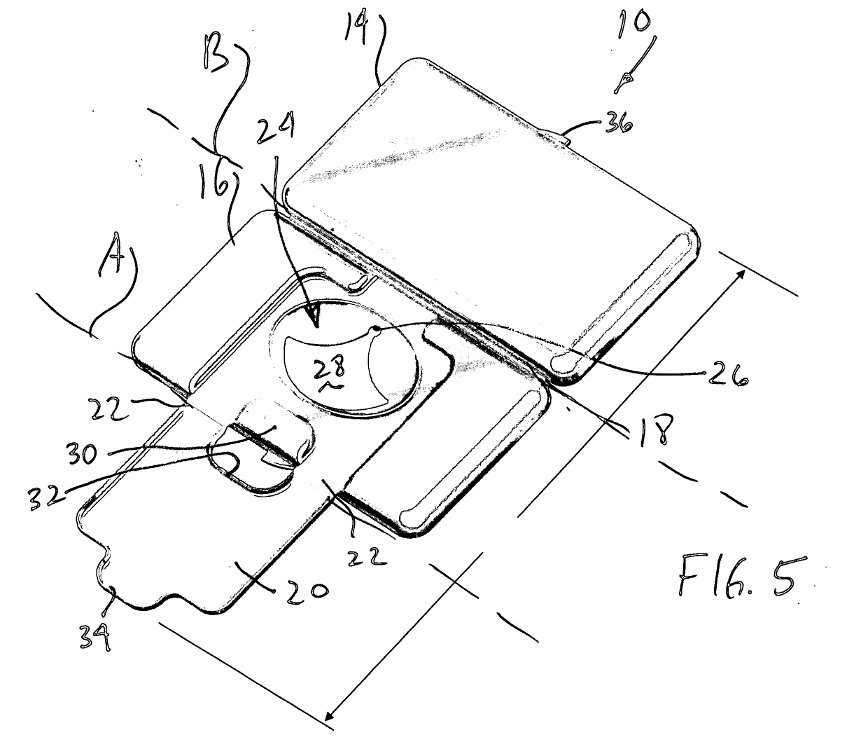

Turning to FIG. 5, the as molded part for box 10 is shown. This advantageously illustrates the single-piece nature of manufacture of this product and further illustrates the substantially parallel nature of axes A and B as discussed above. Of course, the configuration of FIG. 5 also leads to a part which has a relatively large footprint in the mold.

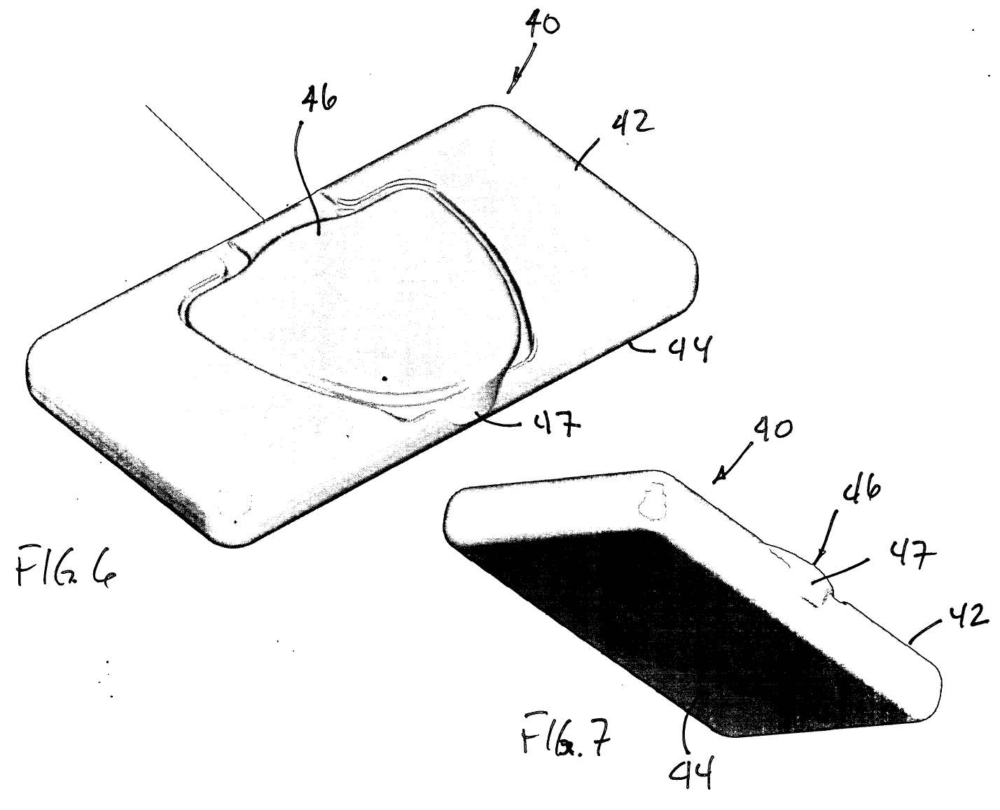

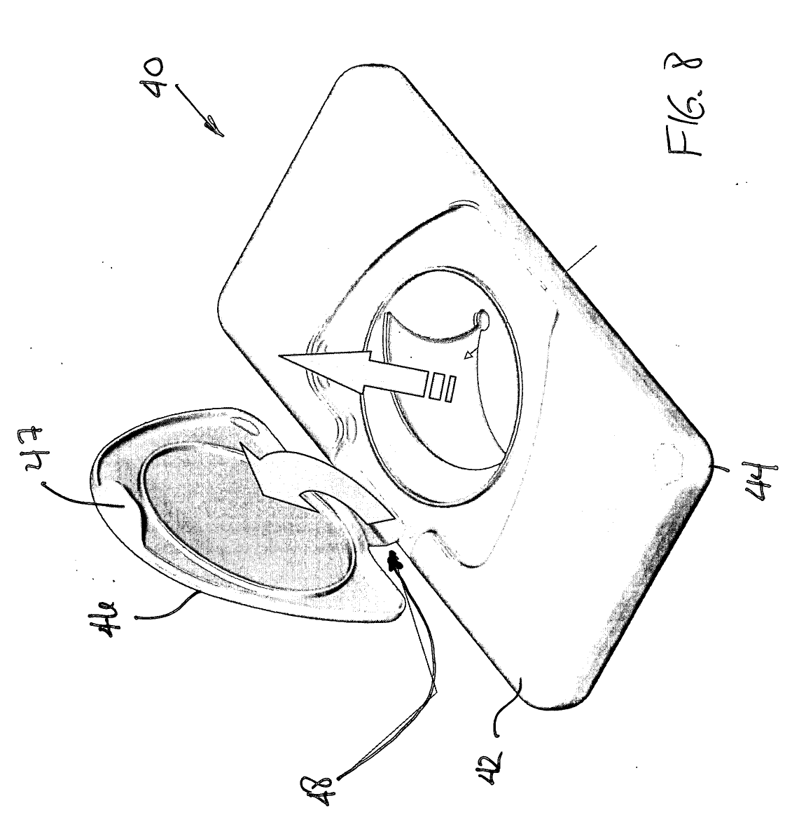

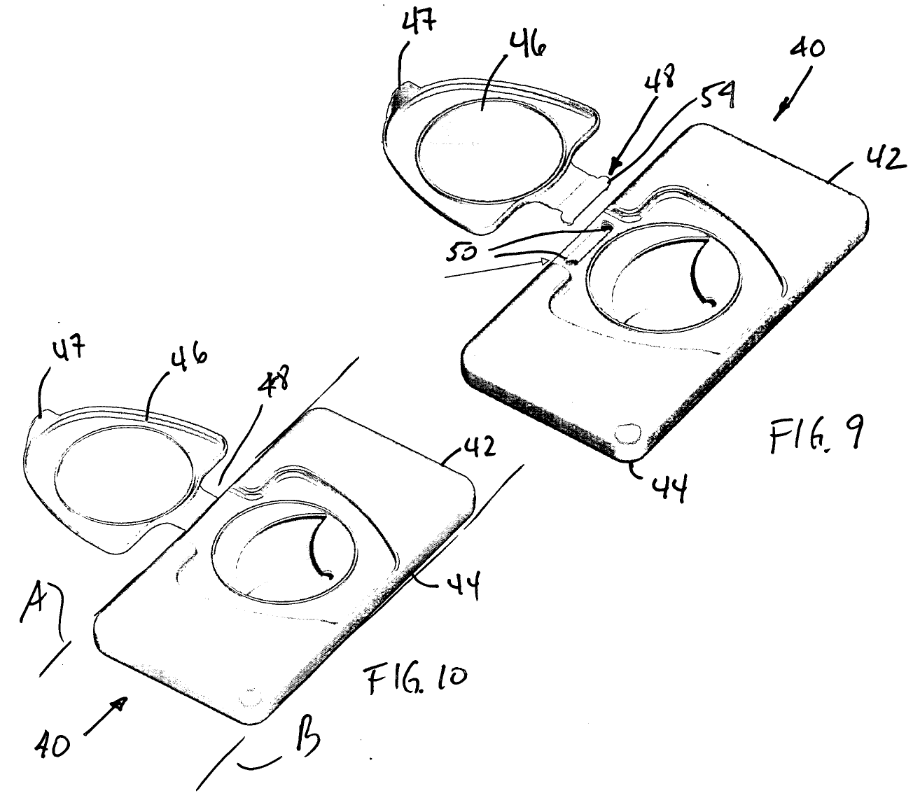

Turning to FIG. 6, an alternative embodiment of the present invention is provided. This embodiment is substantially the same as that of FIG. 1, with the exception being that the tissue hatch or dispensing cover is a separately molded part. Thus, FIG. 6 illustrates a box 40 having an upper body portion 42 and a lower body portion 44 along with a dispensing cover or tissue hatch 46, and these elements are advantageously hingedly mounted relative to each other as discussed above. FIGS. 6 and 7 together also further illustrate a finger lift 47 which is advantageously structured to allow ease in opening of the dispensing cover 46 relative to upper body portion 42.

FIG. 8 illustrates box 40 with cover 46 in an open position for removal of a tissue in a manner which is substantially identical to that of the embodiment of FIG. 1. This figure further illustrates the hinged connection of cover 46 relative to upper body portion 42, in this instance through a living hinge 48. In accordance with the present invention, and as illustrated in FIG. 9, cover member 46 is advantageously molded as a separate part, and is also preferably provided having structure for securing to upper body portion 42 as desired. In the embodiment shown in FIG. 9, cover 46 has a split post detent snap living hinge assembly 48 which is adapted to secure within apurtures 50 or post holes which are positioned in upper body portion 42. As shown in FIG. 9, this structure can include a recessed area 52 on upper body portion 42 which is adapted to match the size and shape of the body portion 54 which carries the split posts of cover 46. This advantageously allows for a substantially permenant mounting of cover 46 to upper body portion 42, after molding, so as to provide a resulting package having the same functionality as that of the embodiment of FIG. 1 while reducing the footprint in the mold of this structure.

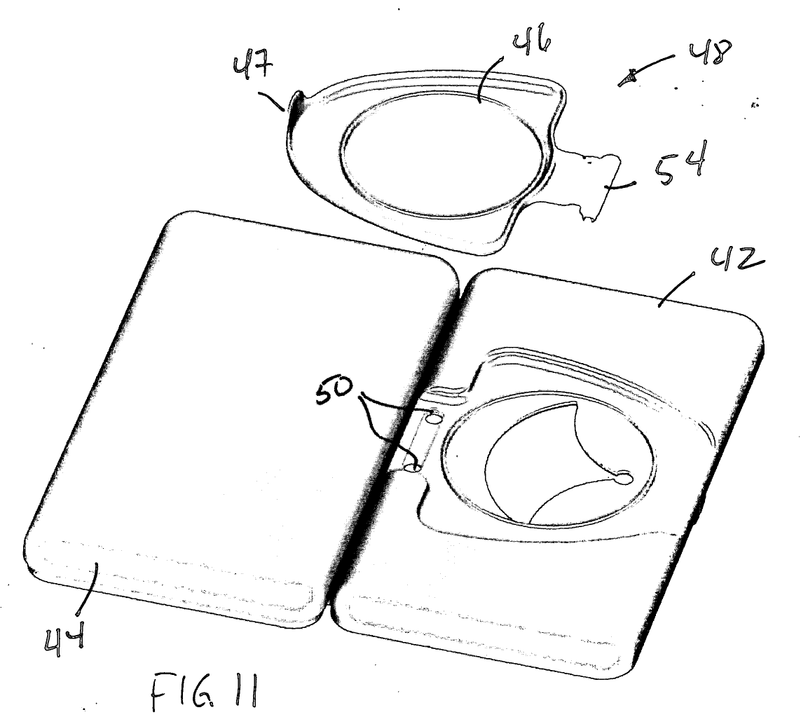

FIG. 10 shows cover 46 after assembly to upper body portion 42, and should make clear that the resulting package functions substantially the same as the embodiment of FIG. 1. That is, cover 46 is hingedly mounted to upper body portion 42 for pivot around an axis A as shown in FIG. 10, and upper body portion is pivotably mounted relative lower body portion 46 for pivot about an axis B also as shown in FIG. 10, which is substantially parallel to and spaced from axis A.

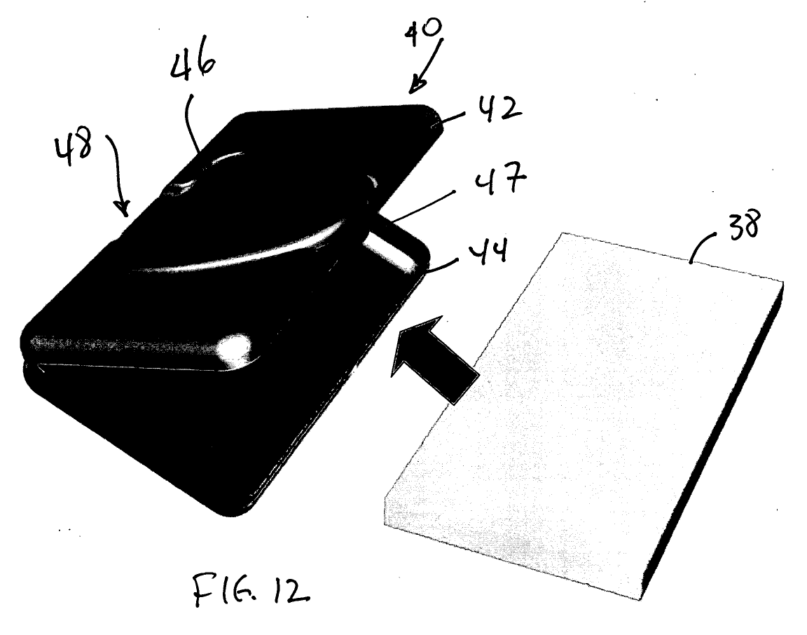

FIG. 11 illustrates the parts of this embodiment of the present invention as molded, and therefore shows one part containing upper body portion 42 and lower body portion 44 and a second part providing cover 46. This results in a substantially reduced footprint as compared to the product of the embodiment of FIG. 1.

FIG. 12 illustrates loading of a tissue stack into box 40 in accordance with the present invention and should make clear the loading of this embodiment is substantially the same as with the embodiment of FIG. 1.

The box in accordance with the present invention can be made from any material which is suitable for the type of product to be held and which would be well known to a person of ordinary skill in the art.

The package can also be provided having dimensions which are suitable for use with various desired sizes and shapes of tissues.

It should of course be appreciated that the above-description and figures are provided as exemplary embodiments of the present invention. Numerous modifications of size, shape, parts and dimensions would be readily apparent to a person of ordinary skill in the art and are considered to be well within the broad

1. A package for dispensing tissues, comprising:
a body having a lower body portion and an upper body portion defining a tissue holding space, wherein the lower body portion is hingedly connected to the upper body portion along a first pivot axis, and wherein the upper body portion has a tissue opening for allowing tissues to be removed from the tissue holding space; and
a tissue access hatch hingedly connected to the body along a second pivot axis which is spaced from and substantially parallel to the first pivot axis.

2. The package of claim 1, wherein the upper body portion has opposed side edges, and wherein a first edge of the side edges is coincident with the first pivot axis and a second edge of the side edges is coincident with the second pivot axis.

3. The package of claim 1, wherein the upper portion defines an inset area, and wherein the tissue access hatch fits within the inset area.

4. The package of claim 1, wherein the upper body portion, the lower body portion and the tissue access hatch are a single molded part.

5. The package of claim 1, wherein the first pivot axis is a living hinge.

6. The package of claim 1, wherein the second pivot axis is a living hinge.

7. The package of claim 6, wherein the second pivot axis comprises two spaced living hinges, and wherein the upper body portion has an upwardly elevated surface defined between the two spaced living hinges.

8. The package of claim 1, wherein the tissue access hatch is a separate molded element with respect to the body.

9. The package of claim 8, wherein the tissue access hatch has a base, and further comprising structure at the upper body portion for fixedly receiving the base relative to the body.

10. The package of claim 9, wherein the base and the structure define a post-detent snap assembly.

11. The package of claim 1, further comprising a body latch along the second edge for holding the upper body portion and the lower body portion closed and a hatch latch along the first edge for holding the tissue access hatch and the upper body portion closed.
